(19) United States
(12) Patent Application Publication
(10) Pub. No.: US 2008/0051357 A1
(43) Pub. Date: Feb. 28, 2008

(54) COMPOSITION FOR ATTENUATING NEUROPATHIC PAIN COMPRISING A RECOMBINANT VENTOR EXPRESSING GAD65

(75) Inventors: Jin Woo Chang, Seoul (KR); Heuiran Lee, Seoul (KR); Boyoung Lee, Seoul (KR); Jaehyung Kim, Seoul (KR); Sung Jin Kim, Chungju (KR)

Correspondence Address: SQUIRE, SANDERS & DEMPSEY L.L.P, PATENT DEPARTMENT, ONE MARITIME PLAZA, SUITE 300, SAN FRANCISCO, CA, 94111-3492, US

(73) Assignees: Industry-Academic Cooperation Foundation, Yonsei University, Seoul (KR); University of Ulsan Foundation for Industry Cooperation, Nam-gu (KR)

(21) Appl. No.: 11/691,478

(22) Filed: Mar. 26, 2007

(30) Foreign Application Priority Data
Jul. 19, 2006 (KR) 10-2006-0067264

(51) Int. Cl. A61K 31/7088; A61P 25/02

(52) U.S. Cl. 514/44

(57) ABSTRACT

Provided is a pharmaceutical composition for attenuation of neuropathic pain comprising a GAD65-expressing recombinant vector. Direct introduction of the pharmaceutical composition of the present invention into a sciatic nerve leads to immediate therapeutic effects on peripheral neuropathic pain with a sustained and constant duration for several months. Therefore, the composition of the present invention is effective for the alleviation of peripheral neuropathic pain.

### BACKGROUND OF THE INVENTION

1. Field of the Invention

The present invention relates to a composition for attenuating neuropathic pain comprising a GAD65-expressing recombinant vector.

2. Description of the Related Art

Peripheral neuropathic pain is clinically common, yet little is known about development of effective therapeutic methods.

Neuropathic pain refers to pain that stems from, or is caused by, primary lesions of various nervous systems or a dysfunction of the nervous systems and may be produced by multiple etiological factors (Bridges D., Br. J. Anaesth., 87: 12-26 (2001)). Peripheral neuropathic pain, characterized by a diverse pathological processes, is accompanied by numerous episodes at different sites and at different times depending upon different disease states (Decosterd I. and Woolf C. J., Pain, 87: 149-158 (2000)). Among intricate and complicated mechanisms underlying neuropathic pain, partial nerve injury appears to cause selective loss of GABAergic inhibitory synaptic current in the spinal cord. This feature contributes to phenotypes of neuropathic pain syndromes (Moore K. A. et al., J. Neurosci., 22: 6724-6731 (2002); Bennett G. J. et al., Neurochemical and anatomical changes in the dorsal horn of rats with an experimental peripheral neuropathy. In: Processing of sensory information in the superficial dorsal horn of the spinal cord, Plenum: New York, 1989, pp. 463-471; and Moore K. et al., Neurosci News, 4: 5-10 (2001)). γ-aminobutyric acid (GABA) produced by glutamic acid dicarboxylase (GAD) is a principal inhibitory neurotransmitter present in the spinal dorsal horn, and also plays an important role in the ventral horn (Todd A. J. and Maxwell D. J., GABA in the mammalian spinal cord. In: GABA in the nervous systems: the view at fifty years (Martin D. L., Olsen R. W., eds), 2000).

Genetic modification of neurons via the introduction of transgenes may be an effective therapy for the treatment of various neurological diseases, such as Alzheimer's disease, Parkinson's disease, and chronic pain syndromes that are intractable due to, or suffer from, little effective therapy. Previous reports suggest that introduction of vector-mediated foreign GAD and the resulting GABA lead to transient relief of neuropathic pain after spinal cord injury (SCI) (Liu J. et al., Mol. Ther. 10: 57-66 (2004)). To date, due to several advantages including long-term effective gene transfer and minimum side effects (Rolling F. and Samulski R. J., Mol. Biotechnol., 3:9-15 (1995); Kaplitt M. G. et al., Nat. Genet., 8:148-154 (1994); Bankiewicz K. S. et al., Exp. Neurol., 144: 147-156 (1997); and Burger C. et al., Hum. Gene Ther., 16: 781-791 (2005)), a human Adeno-associated virus (HAAV)-based vector system has been most widely applied to the nervous system (Mandel R. J. et al., Mol. Ther., 13: 463-483) (2006)). Further, recombinant AAV2 (rAAV2) preferentially transforms the primary neurons of the central nervous system (Bankiewicz K. S. et al., supra; Mandel R. J. et al., J. Neurosci., 18: 4271-4284 (1998); Wang L. et al., Gene Ther., 9: 381-389 (2002); Clark K. R. et al., J. Drug Target, 7: 269-283 (1999); and Burger C. et al., Mol Ther, 10: 302-317 (2004)).

Two isoforms of mammalian GAD were identified and they are encoded by separate genes (Erlander M. G. et al., Neuron, 7: 91-100 (1991)). GAD65 is present in the membrane-bound form at the synapse and largely participates in the production of synaptic GABA for vesicular release. In contrast, GAD67 is distributed throughout cells and is primarily responsible for the production of cytosolic GABA by secretion of GABA through non-vesicular mechanisms (Soghomonian J. J. and Martin D. L., Trends Pharmacol. Sci., 19: 500-505 (1998); Martin D. L. and A. J. T., Mechanisms controlling GABA synthesis and degradation in the brain. In: GABA in the nervous system: the view at fifty years. Williams & Wilkins: Philadelphia, pp. 25-41 (2000); Mi J. et al., J. Neurosci. Res., 57: 137-148 (1999); and Mackie M. et al., Neuroscience, 119: 461-472 (2003)). Moore et al reported that a concentration of GAD65 significantly decreases in the dorsal horn ipsilateral to the nerve injury site in a rat pain model, but expression of GAD67 does not exhibit such a decrease (Moore K A et al., J. Neurosci., 22: 6724-6731 (2002)).

In recent years, Hao et al succeeded in construction of a recombinant Herpes simplex virus (HSV)-based vector encoding human GAD67 (QHGAD67), reporting that subcutaneous administration of QHGAD67 to foot pads of mice alleviates peripheral neuropathic pain (Hao S. et al., Ann. Neurol., 57: 914-918 (2005)). However, the pain relief effects reached to a maximum within several weeks by administration of QHGAD67, the action of which lasted only up to 2 to 5 weeks, and thereafter sharply disappeared.

Therefore, there is a strong and continuous need in the art for the development of a therapeutic agent capable of exhibiting sustained pain-relief effects against peripheral neuropathic pain.

### SUMMARY OF THE INVENTION

Therefore, the present invention has been made in view of the above problems, and it is an object of the present invention to provide a pharmaceutical composition capable of effectively mitigating neuropathic pain.

In accordance with an aspect of the present invention, the above and other objects can be accomplished by the provision of a pharmaceutical composition for attenuating neuropathic pain of a mammal comprising a GAD65-expressing vector.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a gene map of an rAAV-GAD65 vector;

FIG. 2 is a fluorescence microscopic image showing expression profiles of green fluorescent protein (GFP) 3 or 8 weeks after direct injection of rAAV-GFP into L4 and L5 dorsal root ganglions (DRGs) of neuropathic pain model rats;

FIG. 3 is a micrograph (×100) showing immunohistochemical staining results confirming expression of GAD65 in DRGs 8 weeks after injection of rAAV-GAD65 or physiological saline into L4 and L5 DRGs of neuropathic pain model rats;

FIG. 4 is a graph showing effects of rAAV-GAD65 administration on mechanical allodynia in neuropathic pain model rats (non-treated control (●), rAAV-GAD65 administered (∘), rAAV-GFP administered (␣) and saline-administered control (▴), and *: P<0.05 and **: P<0.01);

FIG. 5 is a graph showing effects of rAAV-GAD65 administration on mechanical hyperalgesia in neuropathic pain model rats (non-treated control (●), rAAV-GAD65 administered (∘), rAAV-GFP administered (□) and saline-administered control (▴), and *: P<0.05 and **: P<0.01); and

FIG. 6 is a bar graph showing a concentration of γ-aminobutyric acid (GABA) measured by HPLC in dorsal horns of animals 8 weeks after injection of rAAV-GAD65 or physiological saline into DRGs of neuropathic pain model rats (** :P<0.01).

### DETAILED DESCRIPTION OF THE PREFERRED EMBODIMENTS

The GAD65-expression vector used as an active ingredient of a pharmaceutical composition according to the present invention may be a virus vector (for example, DNA or RNA virus vector) containing a GAD65 gene. Examples of such a virus vector may include Adenovirus vectors and Adeno-associated virus vectors. The Adeno-associated virus vector induces long-term gene expression and is therefore a vector system more suitable for the treatment of pain. The present invention constructed an rAAV-GAD65 vector for pain treatment having a gene map shown in FIG. 1 by the insertion of a GAD65 gene into an Adeno-associated virus vector under the control of a CMV promoter. The thus-constructed rAAV-GAD65 vector exerts sustained pain alleviation effects against neuropathic pain.

Direct injection of the rAAV-GAD65 vector showing a high expression of GAD65 and GABA into dorsal root ganglion (DRGs) of the rat suffering from neuropathic pain due to sciatic nerve injury produces continuous GAD65 expression in DRGs lasting up to more than 8 weeks after injection of the vector, and the resultant GABA release, thereby resulting in significant alleviation of neuropathic pain. Such therapeutic effects immediately manifest and last for several months, during which the effects are substantially constant. Therefore, the composition of the present invention comprising a GAD65-expression vector is highly effective for alleviation of neuropathic pain, particularly peripheral neuropathic pain.

The composition of the present invention may comprise one or more pharmaceutically acceptable carriers and vehicles, and optionally other therapeutically effective ingredients, in addition to the active ingredient GAD65-expression vector. The composition may be sterile and/or may contain adjuvants such as a preservative, a stabilizer, a wetting agent or an emulsification promoter, a salt for osmotic control, and/or a buffer. Further, the composition may be formulated into desired preparations by conventional methods known in the art. For example, a typical parenteral dosage form is an injectable formulation that is preferably provided in the form of an isotonic aqueous solution or a suspension.

The composition of the present invention may be administered to a subject, such as a mammal, e.g. a human, via any suitable route among various parenteral routes, according to gene therapy known to the public. In particular, most effective is administration of the composition to dorsal root ganglions (DRGs) of the subject via direct injection.

The composition may be given at a single dose or an equally divided multiple dose per day as 1 ng to 100 μg/kg BW/day, preferably 10 ng to 10 μg/kg BW/day of the active ingredient GAD-expression vector for a mammal including a human.

However, it is to be understood that a practical dose of the active ingredient will be determined taking into consideration various factors such as severity of pain, the selected administration route, age, sex, weight and conditions of a subject, and the like. Therefore, it will be appreciated that the scope of the present invention is not limited to the above-specified dosage range.

### EXAMPLES

Now, the present invention will be described in more detail with reference to the following Examples. These examples are provided only for illustrating the present invention and should not be construed as limiting the scope and spirit of the present invention.

### Reference Example

### Statistical Analysis

All data were expressed as mean±SEMs. Statistical analysis was carried out using analysis of variance (ANOVA) (electrophysiological recording data) or the Kruskal-Wallis One-way Analysis of Variance, and then the Mann-Whitney U-test was carried out for the comparison of behavior data between each group. Statistical significance was given for P<0.01 and P<0.05. All of statistical analyses were performed using SPSS (version 11.5, SPSS Inc., Chicago, Ill.).

### Example 1

### Construction of Recombinant Adeno-Associated Virus

Adeno-associated virus used in the present invention was constructed and produced based on an AAV helper-free system (available from Stratagene, Kirkland, Wash.).

rAAV2-JDK-GAD65 (hereinafter, referred to as “rAAV-GAD65”) is a construct with the insertion of a gene coding for rat GAD65 into a pJDK plasmid that is an Adeno-associated virus containing a modified CMV promoter (JDK; SEQ. ID. NO.: 1) (Lee B. et al., Gene Ther., 12: 1215-1222 (2005)).

Construction of rAAV-GAD65

First, cDNA of rat GAD65 was synthesized using RT-PCR (reverse transcription-polymerase chain reaction).

The hippocampus was removed from rats and total RNA was extracted from the hippocampus using a Trizol solution (Invitrogen). Using the extracted RNA as a template and a sequence of SEQ. ID. NO.: 2 (5′-GC C̅C̅T̅C̅G̅A̅G̅TTACAAATCTTGTCCCAGGCG-3′; shown with the XbaI recognition site underlined) complementary to 3′-end of GAD mRNA as a primer, cDNA was synthesized. Thereafter, using the synthesized cDNA as a template and the above primer of SEQ. ID. NO.: 2 and a sequence of SEQ. ID. NO.: 3 (5′-GT C̅T̅C̅T̅A̅G̅A̅CCACCATGGCATCTCCGGGCTCTG-3′; shown with the XhoI recognition site) complementary to 5′-end of GAD mRNA as primers, PCR was carried out to obtain a PCR product for rat GAD65. The PCR product was cleaved with XbaI and XhoI and was inserted into pBluescript SK(+) (Stratagene) cleaved with the same restriction enzymes to obtain pBluescript SK-GAD65. pBluescript SK-GAD65 was sequenced to re-confirm whether a nucleotide sequence was correctly incorporated.

In order to construct a pAAV-GAD65 vector containing endogenous GAD65 necessary in production of rAAV-GAD65 virus, the GAD65 gene of pBluescript SK-GAD65 was sub-cloned into a pJDK plasmid (courtesy of Duk-Kyung Kim, M D, PhD, Sungkyunkwan University School of Medicine, Seoul, Korea; Byun J. et al., J. Mol. Cell Cardiol., 33: 295-305 (2001)) for the Adeno-associated virus. That is, the pJDK plasmid and pBluescript SK-GAD65 were respectively cleaved with EcoRI (NEB) and a GAD65 DNA fragment was inserted into the cleaved pJDK plasmid using T4 DNA ligase (Takara Shuzo Co., Ltd., Tokyo, Japan). The resulting plasmid was used to transform E. coli XL-1 Blue competent cells. Transformed cells were cultured on an LB plate containing 50 μg/mL of kanamycin and selected to recover a plasmid pAAV-GAD65. A gene map of plasmid pAAV-GAD65 (rAAV-GAD65 virus was the same) is shown in FIG. 1, wherein TR represents a terminal repeat, P JDK represents a modified CMV promoter, e.g. JDK promoter, and Poly A represents a polyadenylation sequence.

pAAV-GFP contains a humanized Renilla GFP(hrGFP) gene (Stratagene) under the control of a ubiquitous CMV promoter in the backbone of pAAV vector (Stratagene).

Meanwhile, pRepCap and pHelper vectors necessary for the construction of Adeno-associated virus were also purchased from Stratagene.

In order to obtain a pure preparation of rAAVs, 293T cells (courtesy of Dr. J. Jung, Harvard Medical School) cultured on a 10×10 cm dish were transfected with pRepCap or pHelper in conjunction with pAAV-GAD65 or pAAV-GFP using a calcium phosphate-mediated transfection method (Nam Y. R. et al., Oncol. Rep., 12: 761-766 (2004)). This was followed by the single-step column purification (SSCP) (Auricchio A. et al., Hum. Gene Ther., 12: 71-76 (2001)). The purified virus solution was dialyzed against a PBS buffer (PH 7.4), and the purified high-concentration rAAVs were stored in a PBS buffer containing 2% sorbitol at −80□ (Kim S. J. et al., Oncol. Rep., 14: 1475-1479 (2005)).

Total and infectious virus particles were respectively analyzed using an ELISA kit (Progen Inc., Heidelberg, Germany) and immunocytochemistry for GAD65 (Chemicon, Calif.) (Nam Y. R. et al., supra).

### Example 2

### Construction of Animal Model of Neuropathic Pain

8-10 week-old Sprague Dawley male rats weighing 180-200 g were placed in cages each housing five animals. Rats were raised in a breeding room maintained constantly at a temperature of 22±1° C., humidity of 50±5% and a 12 h/12 h light/dark (L/ D) cycle. Animals were given free access to food and drinking water.

Rats were anesthetized with a sodium pentobarbital solution (50 mg/kg) and a segment of the left sciatic nerve was exposed at around the middle part of the femur. The surrounding tissues were carefully removed and the sciatic nerve was carefully fixed by a pincette. Three main parts of the sciatic nerve (tibial, shank and common fibular nerves) were clearly separated under a surgical microscope (Olympus, Japan). In order to construct an effective neuropathic pain model, the tibial and shank nerves were completely severed and firmly ligated and the fibular nerve was left intact. Following hemostatic treatment, the severed region was sutured together with muscle and skin.

### Experimental Example 1

### Transduction Efficiency of rAAVs into DRGs

In order to investigate a transduction efficiency of rAAVs into dorsal root ganglions (DRGs), an animal model (n=6) was surgically established in the same manner as in Example 2 and 2 weeks later a part of the vertebra was removed from the anesthetized rat, thereby surgically exposing ipsilateral lumbar L4 and L5 DRGs. 3 μl (1.3×10⁷ infectious particles/mL) of an rAAV-GFP suspension in physiological saline was transferred to each DRG neurons via a glass micropipette connected to a Hamilton syringe for 20 min and the surgical sites were sutured again.

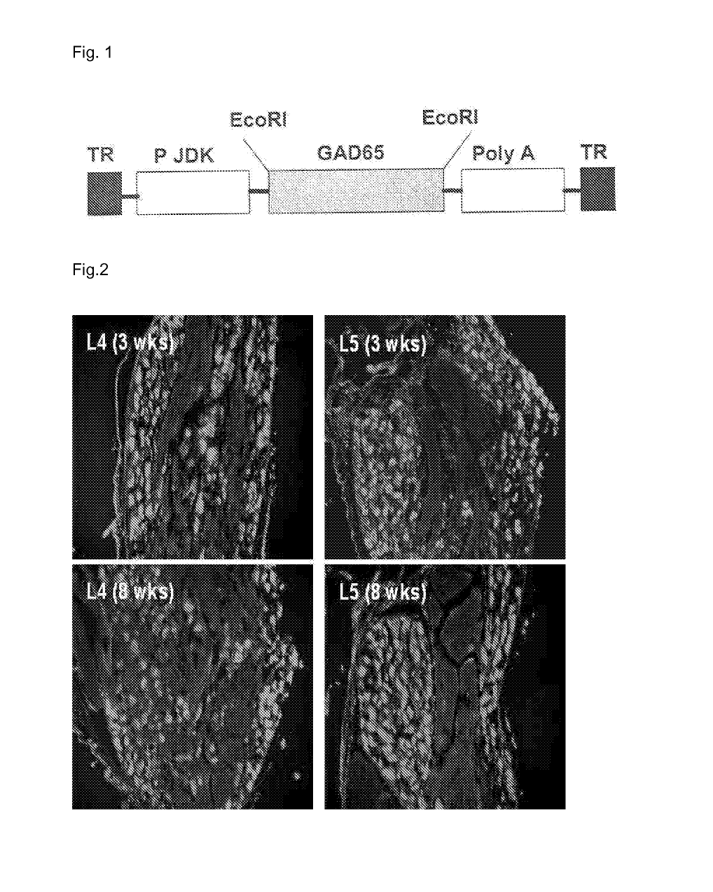

FIG. 2 is a fluorescence microscopic image (×100) showing expression profiles of green fluorescent protein (GFP) 3 or 8 weeks after injection of rAAV-GFP into DRGs of the rats. As can be seen, L4 and L5 DRGs emitted bright fluorescence due to accumulation of GFP after 3 weeks. Further, after introduction of rAAV-GFP into DRGs, GFP was continuously synthesized and a GFP signal was maintained positive at a time point of 8 week without significant changes.

### Example 3

### Examination of GAD65 within DRGs in Neuropathic Pain Model

In order to examine GAD65 within DRGs in a neuropathic pain model, 3 μl of an rAAV-GAD65 suspension in physiological saline (2.4×10⁶ infectious particles/mL) (n=6) or 3 μl (control) (n=5) of physiological saline was directly introduced in the same manner as in Experimental Example 1 into L4 or L5 DRGs of the animal model established as in Example 2.

Rats were sacrificed 8 weeks later and T13-L1 spinal cord and L4-L5 DRGs were isolated, post-fixed, and equilibrated with a 30 % sucrose solution (Lee B. et al., supra) . Thereafter, cryosections were prepared, and each section was stained with polyclonal GAD65 antibodies (Chemicon, Calif.) and then was immunochemically stained with FITC-conjugated secondary antibodies (Santa Cruz Biotech).

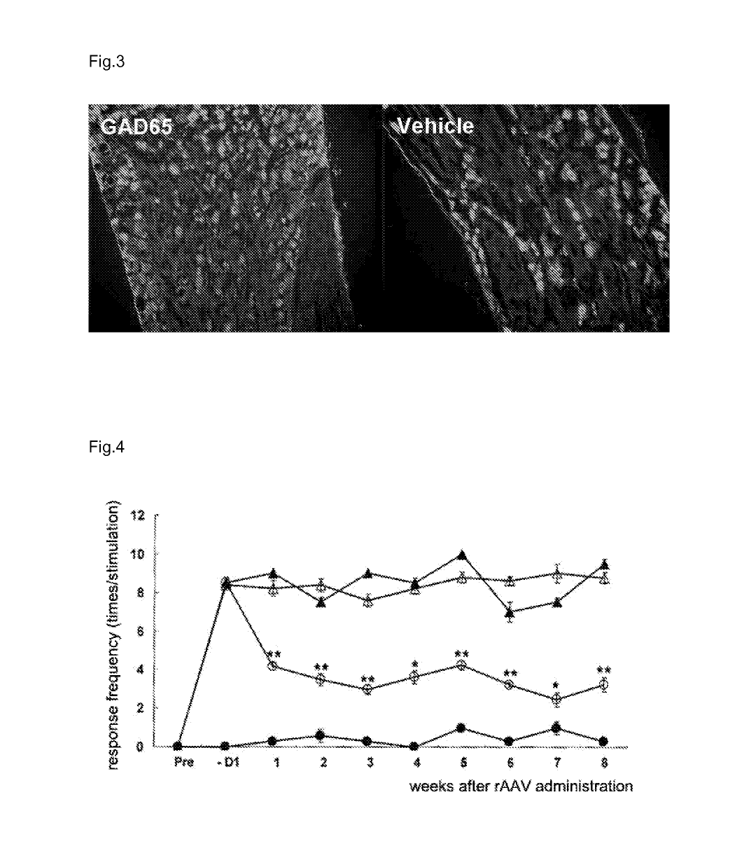

FIG. 3 is a micrograph (×100) showing immunohistochemical staining results of DRGs 8 weeks after administration of rAAV-GAD65. As can be seen, GAD65-specific immunohistochemical staining easily detected expression of GAD65 in DRGs, and the concentration of GAD65 was significantly higher in DRG with injection of rAAV-GAD65, as compared to the control group with an injection of physiological saline.

### Example 4

### Attenuation of Neuropathic Pain-Induced Mechanical Allodynia and Hyperalgesia via Expression of GAD65 in DRGs

(1) Attenuation of Mechanical Allodynia

According to the same manner as in Experimental Example 1, 3 μl of an rAAV-GAD65 suspension in physiological saline (2.4×10⁶ infectious particles/mL) (n=10), 3 μl of an rAAV-GFP suspension in physiological saline (1.3×10⁷ infectious particles/mL) (n=4) or 3 μl (control) (n=5) of physiological saline was directly introduced into L4 or L5 DRGs of the animal model established as in Example 2. Mechanical allodynia of each group was weekly measured from 1 week to 8 weeks by von Frey testing. Non-treated normal rats were employed as a control group.

Rats were housed in acrylic cages (8×10×20 cm) on top of a wire mesh grid which allowed access to the paws. After animals were allowed to acclimate to a new environment for 30 min, non-noxious mechanical stimuli were applied 10 times to side edges of the right and left hind limbs of rats using von Frey filaments (8 mN bending force), and total numbers of both rearing of the right and left hind limbs were measured.

A frequency of hind limb withdrawal at 10-time mechanical stimulation was measured and recorded for four individual groups, an rAAV-GAD65-administered group (n=10), an rAAV-GFP-administered group (n=4), a physiological saline-administered control group (n=7), and a non-treated control group (FIG. 4). One week after administration, the rAAV-GFP-administered group and the physiological saline-administered control group exhibited allodynia response frequency of 8.8±0.2 and 8.5±0.3 per 10 stimulations, respectively. However, the rAAV-GAD65-administered group exhibited a sharp decrease of the allodynia response frequency to a range of 4.2±0.2 per 10 stimulations (a 49.4% decrease relative to the physiological saline-administered control group). More importantly, rAAV-GAD65-administered group continuously exhibited significant alleviation of mechanical allodynia throughout the entire experimental time period. In contrast, the rAAV-GFP-administered group and the physiological saline-administered control group did not exhibit alleviation effects of mechanical allodynia. Further, no mechanical allodynia appeared in the non-treated control group.

(2) Attenuation of Mechanical Hyperalgesia

In order to confirm attenuation of mechanical hyperalgesia by expression of GAD65 in DRGs, an rAAV-GAD65-administered group (n=6), an rAAV-GFP-administered group (n=5), a physiological saline-administered control group (n=7), and a non-treated control group were established as in Section 1, and a pinprick test was carried out using a bent needle (22-gauge) connected to a syringe. In this connection, side edges of paw pads of right and left hind limbs were given pin stimulation having strength enough to evoke reflex withdrawal response also in animals other than the experimental group. Rearing duration was recorded using a stop watch.

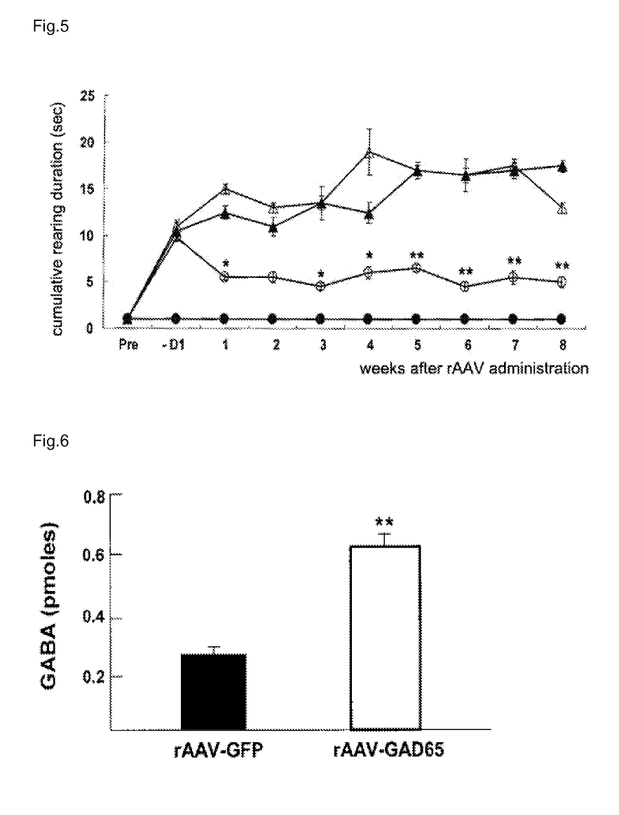

As a result, the rAAV-GAD65-administered group (n=6), the rAAV-GFP-administered group (n=5), and the physiological saline-administered control group (n=7) exhibited significantly increased rearing duration of 9.8±0.4 seconds, 11±0.7 seconds and 10.5±1 seconds, respectively, prior to administration (FIG. 5). Among these groups, only the rAAV-GAD65-administered group exhibited a sharp decrease (5.5±0.4 seconds (P<0.05) in the rearing duration 1 week after injection of viruses; a 44% decrease relative to 12.5±1.0 seconds of the physiological saline-administered control group) with attenuation of symptoms arising from hyperalgesia. Such attenuation effects by rAAV-GAD65 lasted through the entire experimental time period, and both of the rAAV-GFP-administered group and the physiological saline-administered control group did not show hyperalgesia-diminishing effects.

### Example 5

### Determination of Release of GABA from Spinal Dorsal Horn

In order to confirm whether the expression of GAD65 introduced into DRGs leads to a significant increase of GABA in the spinal dorsal horn, a concentration of GABA in the spinal dorsal horn was determined 8 weeks after administration of 3 μl of an rAAV-GAD65 suspension in physiological saline (2.4×10⁶ infectious particles/mL) (n=8) or 3 μl (control) (n=7) of physiological saline to the animal model established as in Example 2, according to the same manner as in Experimental Example 1.

Rats anesthetized with urethane (1.25 mg/kg) were placed on a stereotaxic frame, a backside of the vertebra T13 was exposed and fixed on a horizontal plane using a vertebral clamp. The dura was carefully open and a microdialysis probe (CMA/11, Sweden) was inserted into the vertebra. The probe was perfused with artificial cerebrospinal fluid (CSF) (145 mM NaCl, 2.7 mM KCl, 1.2 mM CaCl₂, 1.0 mM MgCl₂ and 2.0 mM NaH₂PO₄, pH 7.4). A flow rate was carefully adjusted to 1.0 μl/min using a CMA/102 pump (CMA/ microdialysis, Sweden). Thereafter, 30 μl of each cerebrospinal fluid was collected, and mixed with 60 μl of a working solution consisted of 3 mL of OPA mother liquid (2.7 mg of O-phthaldialdehyde, 5 μl of 2-mercaptoethanol and 9 mL of 0.1 M sodium tetraborate in 1 mL of MeOH) and 1 mL of sodium tetraborate, followed by HPLC analysis. The HPLC analysis was carried out using a reverse-phase column (AccQ-Tag, 3.9×150 mm, Waters for amino acid analysis, Ireland) and a 0.02 M sodium acetate buffer (pH 4.6) containing 30% acetonitrile as a mobile phase. Peaks were respectively detected at an excitation wavelength of 340 nm and an emission wavelength of 460 nm, using RF-10Ax1 (Shimazu Corp., Japan) at 30□ and a flow rate of 0.7 mL/min.

As can be seen from FIG. 6, rats of the rAAV-GAD65-administered group exhibited a significant increase in the GABA concentration of the spinal dorsal horn, i.e., 0.619±0.064 pmol/μl (P<0.01), whereas the physiological saline-administered control group showed no such an increase in the GABA concentration (0.284±0.065 pmol/μl).

Direct introduction of a pharmaceutical composition of the present invention comprising a GAD65-expressing vector into a sciatic nerve leads to immediate therapeutic effects on peripheral neuropathic pain with a sustained and constant duration for several months. Therefore, the composition of the present invention is effective for attenuation of peripheral neuropathic pain.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for attenuating neuropathic pain of a mammal, comprising a GAD65-expressing recombinant vector.

2. The composition according to claim 1, wherein the recombinant vector is a recombinant virus vector containing a GAD65 gene.

3. The composition according to claim 2, wherein the recombinant virus vector is a recombinant Adeno-associated virus vector.

4. The composition according to claim 1, wherein the recombinant vector is an rAAV-GAD65 vector having a gene map as shown in FIG. 1.

5. The composition according to claim 1, wherein the neuropathic pain is peripheral neuropathic pain.

6. The composition according to claim 1, wherein the mammal is a human.

7. An injectable preparation comprising of the pharmaceutical composition of claim 1.


SEQUENCE LISTING (3 sequences)

SEQ ID NO: 1 - DNA, 693 bp, Artificial Sequence
  JDK promoter
ggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagc

SEQ ID NO: 2 - DNA, 30 bp, Artificial Sequence
  primer complementary to the 3′-end of GAD mRNA
gccctcgagttacaaatcttgtcccaggcg

SEQ ID NO: 3 - DNA, 33 bp, Artificial Sequence
  primer complementary to the 5′-end of the GAD mRNA
gtctctagaccaccatggcatctccgggctctg